(19) United States
(12) Patent Application Publication
(10) Pub. No.: US 2011/0301504 A1
(43) Pub. Date: Dec. 8, 2011

(54) PRESSURE-DETECTING SHOE

(75) Inventors: HAI LAN, Tu-Cheng (TW); GA-LANE CHEN, Santa Clara, CA (US)

(73) Assignee: HON HAI PRECISION INDUSTRY CO., LTD., Tu-Cheng (TW)

(21) Appl. No.: 12/860,919

(22) Filed: Aug. 22, 2010

(30) Foreign Application Priority Data
Jun. 4, 2010 (TW) 99118142

(51) Int. Cl. A61B 5/103

(52) U.S. Cl. 600/592

(57) ABSTRACT

A pressure-detecting shoe includes a main body and a pressure sensor module. The main body includes a toe region, a ball region and a heel region. The pressure sensor module includes a toe pressure sensitive bag, a toe pressure sensor, a ball pressure sensitive bag, a ball pressure sensor, a heel pressure sensitive bag, a heel pressure sensor, a process unit, and a power supply unit supplying electric power for the process unit. The toe pressure sensitive bag, the ball pressure sensitive bag and the heel pressure sensitive bag are impressed by the toe region, the ball region and the heel region respectively. The toe, ball and heel pressure sensors detect corresponding pressures from the toe region, the ball region and the heel region, respectively, and transform the corresponding pressures to pressure signals. The process unit obtains the corresponding pressure signals and calculates the corresponding pressures according to the signals.

### BACKGROUND

1. Technical Field

The present disclosure relates to shoes, and particularly, to a pressure-detecting shoe.

2. Description of Related Art

A shoe is an item of footwear for protecting the human foot in daily lives, such as in jogging, racing, physical training, dancing, or other forms of competition. In order to increase the wearing comfort, materials and structures of shoes are diversified. For example, high permeability materials or elastic gasbags may be applied to the related shoes.

However, such shoes can not provide feedback of the wear and tear of the shoe, and thus do not provide the valuable information to healthcare providers. For example, patients with an injured foot or leg, or patients having trouble moving their foot or leg due to an injury or disease, may have to undergo physical therapy for recovery. Accurate information in the way the patient walks or carries themselves may be an important part of recovery. Since the above mentioned shoes can not provide feedback during the recovery period, the related information can only be obtained through observation by doctors, where some information may be overlooked or difficult to ascertain. Besides the medical use, the information in daily walking, running, dancing, video game playing or other activities of a normal, is also helpful to judge or correct the user's activities, posture or stride.

Accordingly, it is desirable to provide a shoe which can provide feedback while under the care of a healthcare professional.

### BRIEF DESCRIPTION OF THE DRAWINGS

Many aspects of the disclosure can be better understood with reference to the drawings. The components in the drawings are not necessarily drawn to scale, the emphasis instead being placed upon clearly illustrating the principles of the present disclosure. Moreover, in the drawings, like reference numerals designate corresponding parts throughout the views.

FIG. 1 is a schematic view of a pressure-detecting shoe according to a first embodiment of the present disclosure, the pressure-detecting shoe including a main body and a pressure sensor module.

FIG. 2 is a schematic view of the main body and the pressure sensor module of the pressure-detecting shoe of FIG. 1.

FIG. 3 is a schematic view of a main body and a pressure sensor module of a pressure-detecting shoe according to a second embodiment of the present disclosure.

### DETAILED DESCRIPTION

Embodiments of the disclosure will now be described in detail with reference to the accompanying drawings.

The present disclosure relates to a pressure-detecting shoe having a micro-electro-mechanical system (MEMS), which integrates micro-sensors, actuators, signal processing circuits, control circuits, interface circuits, with a communication and power supply therein. The pressure sensors of the present disclosure may be any type of

MEMS pressure sensor, such as piezoresistive MEMS pressure sensors or capacitive MEMS pressure sensors.

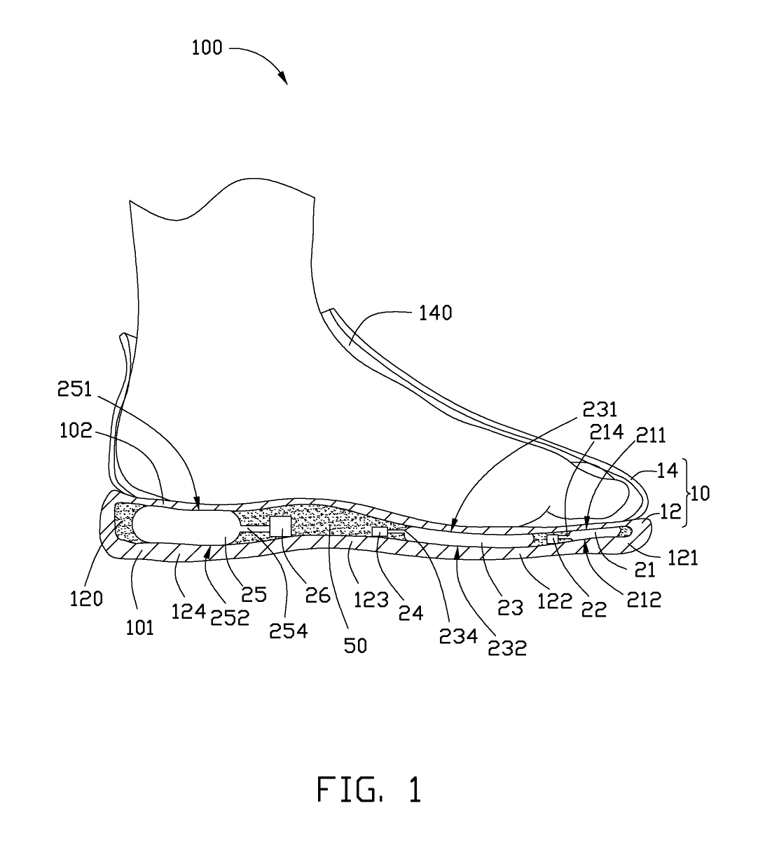

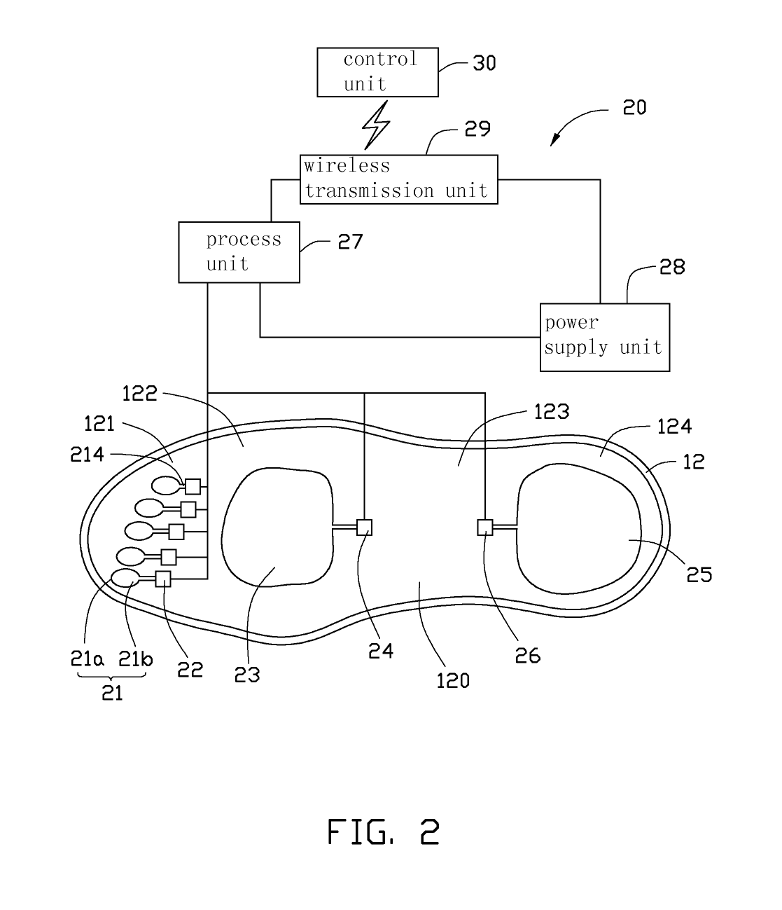

As shown in FIG. 1 and FIG. 2, a first embodiment of a pressure-detecting shoe 100 includes a main body 10 and a pressure sensor module 20.

The main body 10 includes a sole 12 and a vamp 14. The sole 12 defines a toe region 121, a ball region 122 adjacent to the toe region 121, an arch region 123 adjacent to the ball region 122 and a heel region 124 adjacent to the arch region 123. The sole 12 includes an outsole 101 and an insole 102. The outsole 101 and the insole 102 define a housing space 120 there between. The sole 12 and the vamp 14 define a wearing space 140 there between to accommodate a user's foot.

The pressure sensor module 20 is located in the housing space 120, and includes a plurality of toe pressure sensitive bags 21 corresponding to the toe region 121, a plurality of toe pressure sensors 22 connected to the toe pressure sensitive bags 21, a ball pressure sensitive bag 23 corresponding to the ball region 122, a ball pressure sensor 24 connected to the ball pressure sensitive bag 23, a heel pressure sensitive bag 25 corresponding to the heel region 124, a heel pressure sensor 26 connected to the heel pressure sensitive bag 25, a process unit 27 and a power supply unit 28 to supply electric power to the process unit 27.

The pressure sensor module 20 may include five toe pressure sensitive bags 21 corresponding to five toes respectively in the toe region 121, and five toe pressure sensors 22 connected to the five toe pressure sensitive bags 21 respectively in this embodiment. The pressure sensor module 20 may include only one toe pressure sensitive bag 21 corresponding to the five toes and one toe pressure sensor 22, or five toe pressure sensitive bags 21 and only one toe pressure sensor 22 in other embodiments.

The toe sensor 22 is connected to the toe pressure sensitive bag 21 for sensing pressure applied to the toe pressure sensitive bag 21 and correspondingly providing a signal. Specifically speaking, each of the toe pressure sensitive bags 21 includes a first compressible surface 211 and a first fixed surface 212 opposite to the first compressible surface 211, and the first compressible surface 211 and the first fixed surface 212 are attached to the insole 102 and the outsole 101 respectively. Each of the toe pressure sensitive bags 21 includes a bag body 21a and a fluid 21b therein. The fluid 21b may be gas or liquid with a predetermined volume. The bag body 21a defines an opening 214, and the toe pressure sensor 22 seals the opening 214 so as to sense the pressure applied to the toe pressure sensitive bag 21 according to the fluid pressure of the fluid 21b. The toe pressure sensitive bags 21 are made out of elastic materials, such as rubber.

When a force is applied to the insole 102 in the toe region 121, the corresponding pressure is applied to at least one of the toe pressure sensitive bags 21 and compresses the first compressible surface 211. The toe pressure sensitive bag 21 is compressed, and a portion of the fluid 21b flows outward through the opening 214. The gas flow or liquid flow affects the corresponding toe pressure sensor 22. The toe pressure sensor 22 can sense the pressure applied to the toe pressure sensitive bag 21 from the gas flow or liquid flow of the fluid 21b, and provides a corresponding signal. The value and producing timing of the digital signal can show the magnitude and applied timing of the pressure. When there is no force applied to the insole 102 in the toe region 121, the toe bag 21 decompresses and returns to its normal shape.

The toe pressure sensors 22 can be located in the housing space 120 corresponding to the toe region 121 in this embodiment. It is noted that the positions of the toe pressure sensors 22 should not be limited thereto, and the toe pressure sensors 22 may be located in any position. For instance, the toe pressure sensors 22 may be located in the ball region 122, the arch region 123 or the heel region 124 in other embodiments.

The ball sensor 24 is connected to the ball pressure sensitive bag 23 for sensing pressure applied to the ball pressure sensitive bag 23 and correspondingly providing a signal. The ball pressure sensitive bag 23 includes a second compressible surface 231 and a second fixed surface 232 opposite to the second compressible surface 231, and the second compressible surface 231 and the second fixed surface 232 are attached to the insole 102 and the outsole 101 respectively. The ball pressure sensitive bag 23 is filled with a fluid. The fluid may be gas or liquid with a predetermined volume. The ball pressure sensitive bag 23 defines an opening 234, and the ball pressure sensor 24 seals the opening 234 so as to sense the pressure applied to the ball pressure sensitive bag 23 according to the fluid pressure of the fluid filled in the ball pressure sensitive bag 23. The ball pressure sensitive bag 23 is made out of elastic material, such as rubber.

The heel pressure sensor 26 is connected to the heel pressure sensitive bag 25 for sensing pressure applied to the heel pressure sensitive bag 25 and correspondingly providing a signal. The heel pressure sensitive bag 25 includes a third compressible surface 251 and a third fixed surface 252 opposite to the third compressible surface 251, and the third compressible surface 251 and the third fixed surface 252 are attached to the insole 102 and the outsole 101 respectively. The heel pressure sensitive bag 25 is filled with a fluid. The fluid may be gas or liquid with a predetermined volume. The heel pressure sensitive bag 25 defines an opening 254, and the heel pressure sensor 26 seals the opening 254 so as to sense the pressure applied to the heel pressure sensitive bag 25 according to the fluid pressure of the fluid filled in the heel pressure sensitive bag 25. The heel pressure sensitive bag 25 is made out of elastic material, such as rubber.

The process unit 27 is electrically connected to the toe pressure sensor 22, the ball pressure sensor 24, and the heel pressure sensor 26 to receive the signals applied to the toe bag 21, the ball pressure bag 23 and the heel bag 25 respectively. The process unit 27 can be electrically connected to the pressure sensors 22, 24, and 26 through any interconnections. The process unit 27 calculates the pressures applied to the toe bag 21, the ball bag 23, and the heel bag 25 according to the received signals. The process unit 27 may be a micro control unit (MCU) or an application specific integrated circuit (ASIC).

The pressure sensor module 20 may further include a wireless transmission unit 29 and a control unit 30. The wireless transmission unit 29 is electronically connected to the process unit 27 for transmitting data between the control unit 30 and the process unit 27. The wireless transmission unit 29 can receive data from the control unit 30 and transmits data to the control unit 30. The wireless transmission unit 29 may be a BLUETOOTH unit in this embodiment. The power supply unit 28 may be a battery unit, and may supply electric power to both the process unit 27 and the wireless transmission unit 29.

The control unit 30 may be located in a computer or a video game console. The control unit 30 receives the signals from the process unit 27, and may output the signals to a display unit or an executive unit (not shown) of the computer or the video game console. Accordingly, the moving information, such as the stride timing, pressure sensor sequence, compression, compression variations, stride interval of the toe, sole and heel is obtained, and is helpful to the medical professional, patient, walker, runner, dancer and video game player. With this information, the medical professional and patient may acquire recovering status of the patient in detail from the accumulated information. A normal would be able to change their stride while walking or running according to the accumulated information to improve the physical training effect or relaxation of their activities. The dancer and video game player may clearly know their accuracy of the stride timing and movement so as to improve their accuracy in concordance with the music rhythm. The detailed accuracy may provide a greater fun in the game.

For example, the stride of a average person walking is usually the heel, the ball and the toes for one foot, and then the person pushes off to the next step with the toes. However, patients with an injury or disease may have trouble with their stride. For example, they may use the ball to touch the ground before the heel, or their ball and heel may touch the ground simultaneously. In addition, the patients may exhibit a weaker transition force from foot to foot; and stride frequency by comparison. Accordingly, the doctors can compare the actual treading portions, compression forces, and the stride frequencies with average conditions, to diagnose the recovering speed and to determine physical therapy changes or treatment.

The pressure-detecting shoe 100 may optionally include soft stuff 50 located in spaces between the toe bags 21, the toe pressure sensors 22, the ball bag 23, the ball pressure sensor 24, the heel bag 25 and the heel pressure sensor 26 in the housing space 120. The soft stuff 50 can be a buffer to absorb the external shock, and help to hold the pressure sensor module 20.

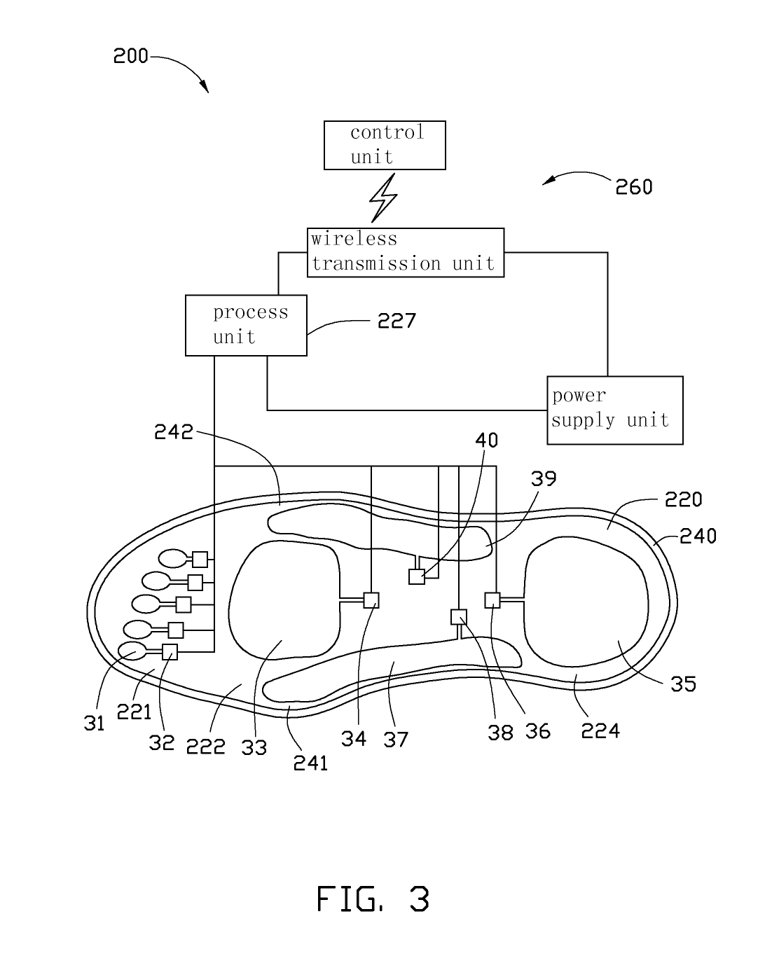

As show in FIG. 3, a second embodiment of the pressure-detecting shoe 200 is similar to the first embodiment of the pressure-detecting shoe 100, except that the sole 240 further defines an inner side region 241 and an outer side region 242, and the pressure sensor module 260 further includes an inner side pressure sensitive bag 37, an inner side pressure sensor 38, an outer side pressure sensitive bag 39 and an outer side pressure sensor 40. The inner side region 241 and the outer side region 242 are located between the toe region 221 and the heel region 224. The inner side region 241 and the outer side region 242 are located on two opposite sides of the ball region 222 respectively.

The inner and outer side pressure sensitive bags 37 and 39 are located in the housing space 220 corresponding to the inner and outer side regions 241 and 242 respectively. The inner and outer side pressure sensors 38 and 40 are connected to the inner and outer side pressure sensitive bags 37 and 39 respectively for sensing the pressure applied to the inner and outer side pressure sensitive bags 37 and 39 and correspondingly providing signals respectively. The structures, arrangements and sensing processes of the inner and outer side pressure sensitive bags 37 and 39 and inner and outer side pressure sensors 38 and 40 are similar to the toe pressure sensitive bag 31 and the toe pressure sensors 32.

The process unit 227 is electrically connected to the toe pressure sensor 32, the ball pressure sensor 34, the inner side pressure sensor 38, the outer side pressure sensor 40 and the heel pressure sensor 36 to receive the signals applied to the toe pressure sensitive bags 31, the ball pressure sensitive bag 33, the inner side pressure sensitive bag 37, the outer side pressure sensitive bag 39 and the heel pressure sensitive bag 35 respectively. Since the pressure sensor module 260 further includes the inner side pressure sensor 38, and the outer side pressure sensor 40, the lateral treading information in both sides of the ball region 222 are obtained.

For example, some dancers not only need to apply weight to the toes, ball and heels of feet, but also needs to apply lateral weight distribution to the inner sides and outer sides of the feet. In such a case, the pressure-detecting shoe 200 provides additional information about the movement and weight distribution on the feet and thus provides greater fun.

The toe bag, the ball bag, the heel bag, the inner side bag, and the outer side bag may include no opening in other embodiments. The toe pressure sensor, the ball pressure sensor, the heel pressure sensor, the inner side pressure sensor, and the outer side pressure sensor may be directly connected to the toe bag, the ball bag, the heel bag, the inner side bag, and the outer side bag respectively. When the toe bag, the ball bag, the heel bag, the inner side bag, or the outer side bag is compressed, the pressure is transmitted to the corresponding pressure sensor directly through the bag body.

The information acquired from the wearing of these shoes have broader application than that of medical use and the accumulation of data for therapy; they can also be used for evaluating activities such as running, dancing or other activities where the user's weight balance and general foot movements would need to be monitored.

What is claimed is:

1. A pressure-detecting shoe, comprising:
a main body, comprising:
a sole, the sole defining a toe region, a ball region adjacent to the toe region, an arch region adjacent to the ball region and a heel region adjacent to the arch region, the sole comprising an outsole and an insole, the outsole and the insole defining a housing space there between; and
a vamp, the vamp and the insole defining a wearing space there between; and
a pressure sensor module located in the housing space, comprising:
a plurality of toe pressure sensitive bags corresponding to the toe region;
a plurality of toe pressure sensors, each pressure sensor being connected to a corresponding toe pressure sensitive bag for sensing a first pressure applied to the corresponding toe pressure sensitive bag and correspondingly providing a first signal;
a ball pressure sensitive bag corresponding to the ball region;
a ball pressure sensor connected to the ball pressure sensitive bag for sensing a second pressure applied to the ball pressure sensitive bag and correspondingly providing a second signal;
a heel pressure sensitive bag corresponding to the heel region;
a heel pressure sensor connected to the heel pressure sensitive bag for sensing a third pressure applied to the heel pressure sensitive bag and correspondingly providing a third signal;
a process unit electrically connected to the toe, ball and heel pressure sensors and configured to receive the first, second and third signals and calculate the first, second and third pressures according to the first, second and third signals; and
a power supply unit configured to supply electric power to the process unit.

2. The pressure-detecting shoe of claim 1, wherein each toe pressure sensitive bag comprises a bag body and a fluid therein, the bag body defines a first opening, and the toe pressure sensor seals the first opening so as to sense the first pressure according to a fluid pressure of the fluid.

3. The pressure-detecting shoe of claim 1, wherein the ball pressure sensitive bag defines a second opening and is filled with a fluid, and the ball pressure sensor seals the second opening so as to sense the second pressure according to a fluid pressure of the fluid filled in the ball pressure sensitive bag.

4. The pressure-detecting shoe of claim 1, wherein the heel pressure sensitive bag defines a third opening and is filled with a fluid, and the heel pressure sensor seals the third opening so as to sense the third pressure according to a fluid pressure of the fluid filled in the heel pressure sensitive bag.

5. The pressure-detecting shoe of claim 1, wherein the toe pressure sensitive bag comprises a first compressible surface and a first fixed surface opposite to the first compressible surface, and the first compressible surface and the first fixed surface are attached to the insole and the outsole respectively.

6. The pressure-detecting shoe of claim 1, wherein the ball pressure sensitive bag comprises a second compressible surface and a second fixed surface opposite to the second compressible surface, and the second compressible surface and the second fixed surface are attached to the insole and the outsole respectively.

7. The pressure-detecting shoe of claim 1, wherein the heel pressure sensitive bag comprises a third compressible surface and a third fixed surface opposite to the third compressible surface, and the third compressible surface and the third fixed surface are attached to the insole and the outsole respectively.

8. The pressure-detecting shoe of claim 1, wherein the pressure sensor module further comprises a wireless transmission unit and a control unit, the wireless transmission unit is electrically connected to the process unit, the wireless transmission unit is configured to receive data from the control unit and emit data to the control unit.

9. The pressure-detecting shoe of claim 8, wherein the wireless transmission unit is a BLUETOOTH unit.

10. The pressure-detecting shoe of claim 1, wherein the pressure sensor module comprises five toe pressure sensitive bags and five toe pressure sensors.

11. The pressure-detecting shoe of claim 1, further comprising a soft stuff located in spaces between the toe pressure sensitive bags, the toe sensors, the ball pressure sensitive bag, the ball pressure sensor, the heel pressure sensitive bag and the heel sensor.

12. The pressure-detecting shoe of claim 1, wherein the sole further defines an inner side region and an outer side region located between the toe region and the heel region, and located on two opposite sides of the ball region respectively.

13. The pressure-detecting shoe of claim 12, wherein the pressure sensor module further comprises:
an inner side pressure sensitive bag located in the housing space corresponding to the inner side region; and
an inner side pressure sensor connected to the inner side pressure sensitive bag for sensing a fourth pressure applied to the inner side pressure sensitive bag and correspondingly providing a fourth signal.

14. The pressure-detecting shoe of claim 13, wherein the pressure sensor module further comprises:
an outer side pressure sensitive bag located in the housing space corresponding to the outer side region; and
an outer side pressure sensor connected to the outer side pressure sensitive bag for sensing a fifth pressure applied to the outer side pressure sensitive bag and correspondingly providing a fifth signal.

15. The pressure-detecting shoe of claim 14, wherein the process unit is electrically connected to the inner side and outer side pressure sensors to receive the fourth and fifth signals and to calculate the fourth and fifth pressures according to the fourth and fifth signals.

16. The pressure-detecting shoe of claim 13, wherein the inner side pressure sensitive bag comprises a bag body and a fluid therein, the bag body defines a fourth opening, and the inner side pressure sensor seals the fourth opening so as to sense the fourth pressure according to a fluid pressure of the fluid.

17. The pressure-detecting shoe of claim 14, wherein the outer side pressure sensitive bag comprises a bag body and a fluid therein, the bag body defines a fifth opening, and the outer side pressure sensor seals the fifth opening so as to sense the fifth pressure according to a fluid pressure of the fluid.

18. A pressure-detecting shoe, comprising:
a sole having a toe region, a ball region adjacent to the toe region, an arch region adjacent to the ball region and a heel region adjacent to the arch region, and defining a housing space;
a plurality of toe pressure sensitive bags received in the housing space and located in the toe region;
a plurality of toe pressure sensors, each pressure sensor being connected to a corresponding toe pressure sensitive bag for sensing a first pressure applied to the corresponding toe pressure sensitive bag and correspondingly providing a first signal;
a ball pressure sensitive bag received in the housing space and located in the ball region;
a ball pressure sensor connected to the ball pressure sensitive bag for sensing a second pressure applied to the ball pressure sensitive bag and correspondingly providing a second signal;
a heel pressure sensitive bag received in the housing space and located in the heel region;
a heel pressure sensor connected to the heel pressure sensitive bag for sensing a third pressure applied to the heel pressure sensitive bag and correspondingly providing a third signal; and
a process unit electrically connected to the toe, ball and heel pressure sensors, the process unit being configured to receive the first, second and third signals, and to calculate the first, second and third pressures according to the first, second and third signals.

19. The pressure-detecting shoe of claim 18, wherein the sole has an inner side region and an outer side region located between the toe region and the heel region, and located on two opposite sides of the ball region respectively; the pressure-detecting shoe comprises an inner side pressure sensitive bag and an inner side pressure sensor, the inner side pressure sensitive bag is received in the housing space and located in the inner side region, the inner side pressure sensor is connected to the inner side pressure sensitive bag for sensing a fourth pressure applied to the inner side pressure sensitive bag and correspondingly providing a fourth signal.

20. The pressure-detecting shoe of claim 18, wherein the sole has an inner side region and an outer side region located between the toe region and the heel region, and located on two opposite sides of the ball region respectively; the pressure-detecting shoe comprises an outer side pressure sensitive bag and an outer side pressure sensor, the outer side pressure sensitive bag is received in the housing space and located in the outer side region, the outer side pressure sensor is connected to the outer side pressure sensitive bag for sensing a fifth pressure applied to the outer side pressure sensitive bag and correspondingly providing a fifth signal.
